(19) United States
(12) Patent Application Publication
(10) Pub. No.: US 2026/0100257 A1
(43) Pub. Date: Apr. 9, 2026

(54) Augmenting Knowledge Not Available in Electronic Health Record System Patient Charts

(71) Applicant: Oracle International Corporation, Redwood Shores, CA (US)

(72) Inventors: Amitabh Saikia, Mountain View, CA (US); Natalee Agassi, Blue Bell, PA (US); Raman Grover, Charlotte, NC (US); Vadim Khotilovich, Leawood, KS (US); Jigar Shirish Patel, Lenexa, KS (US); Raefer Christopher Gabriel, San Mateo, CA (US); Amy Marie Willson, East Aurora, NY (US); Suhas Srinivas Uliyar, Woodinville, WA (US)

(73) Assignee: Oracle International Corporation, Redwood Shores, CA (US)

(21) Appl. No.: 19/294,641

(22) Filed: Aug. 8, 2025

Related U.S. Application Data
(60) Provisional application No. 63/705,415, filed on Oct. 9, 2024.

(51) Int. Cl. G16H 10/60; G16H 70/20

(52) U.S. Cl. CPC G16H 10/60; G16H 70/20

(57) ABSTRACT

A system processes clinical guidance to enhance patient care management. The system receives clinical guidance in digital text form from authoritative medical sources. A large language model analyzes the received clinical guidance to extract key information and relationships. The system generates a structured pathway based on the analyzed clinical guidance. The generated pathway represents a comprehensive summary for a specific disease state, organized into logical steps. These steps encompass treatment goals, management strategies, and measures for preventing complications. The system integrates the generated pathway information into an electronic health record (EHR) system. Within the EHR, the system produces a patient chart incorporating the derived pathway information.

### INCORPORTION BY REFERENCE; DISCLAIMER

The following application is hereby incorporated by reference: application no. 63/705,415, filed on Oct. 9, 2024, entitled “Augmenting Knowledge Not Available In Electronic Health Record System Patient Charts”. The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to electronic health record (EHR) systems.

### BACKGROUND

Electronic health record (EHR) systems serve as digital repositories for patient health information. EHR systems store and manage a wide range of patient data, including medical histories, diagnoses, medications, treatment plans, immunization dates, allergies, radiology images, and laboratory test results. The systems provide real-time, patient-centered records that make information available instantly and securely to authorized users. EHR systems implement security measures to protect sensitive patient data, including encryption, access controls, and audit trails. The systems support interoperability standards, enabling seamless data exchange between different healthcare providers and organizations.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system for augmenting EHR patient charts in accordance with one or more embodiments;

FIG. 2 illustrates an example set of operations for augmenting EHR patient charts in accordance with one or more embodiments;

FIG. 3 illustrates an example of generating structured care recommendations by processing diabetes management clinical guidance; and

FIG. 4 shows a block diagram that illustrates a system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. PATIENT RECORD AUGMENTATION ARCHITECTURE
3. AUGMENTING ELECTRONIC HEALTH RECORD PATIENT CHARTS
4. DIABETES MANAGEMENT PATHWAY GENERATION EXAMPLE
5. ADDITIONAL INFORMATION
6. PRACTICAL APPLICATIONS, ADVANTAGES & IMPROVEMENTS
7. MISCELLANEOUS; EXTENSIONS
8. HARDWARE OVERVIEW

### 1. GENERAL OVERVIEW

One or more embodiments transform clinical guidance into structured pathways for improved patient care. A system receives clinical guidance in digital text form and employs a large language model (LLM) to generate a pathway. The generated pathway represents a structured summary for a specific disease state, organized into logical steps that encompass treatment goals, management strategies, and complication prevention measures. The system then produces a patient chart in an electronic health record (EHR) system, incorporating information derived from the pathway.

One or more embodiments enhance pathway generation through contextual information. The system provides the LLM with clinical guidance alongside contextual data, including pathway templates, examples, and standards. This contextual information guides the LLM in producing more relevant and tailored pathways. The system also generates non-pathway data using the LLM, supplementing the EHR with additional relevant medical information beyond the structured pathway.

One or more embodiments standardize pathway information. The system codifies the generated pathway using standard medical terminologies, assigning specific codes to medical concepts. This standardization facilitates interoperability and consistent interpretation across different healthcare systems.

One or more embodiments personalize care recommendations based on patient-specific data. The system receives patient information from the EHR, parses this information using the LLM, and generates care recommendations by comparing the parsed patient data with the established pathways.

One or more embodiments described in this Specification and/or recited in the claims may not be included in this General Overview section.

### 2. PATIENT RECORD AUGMENTATION ARCHITECTURE

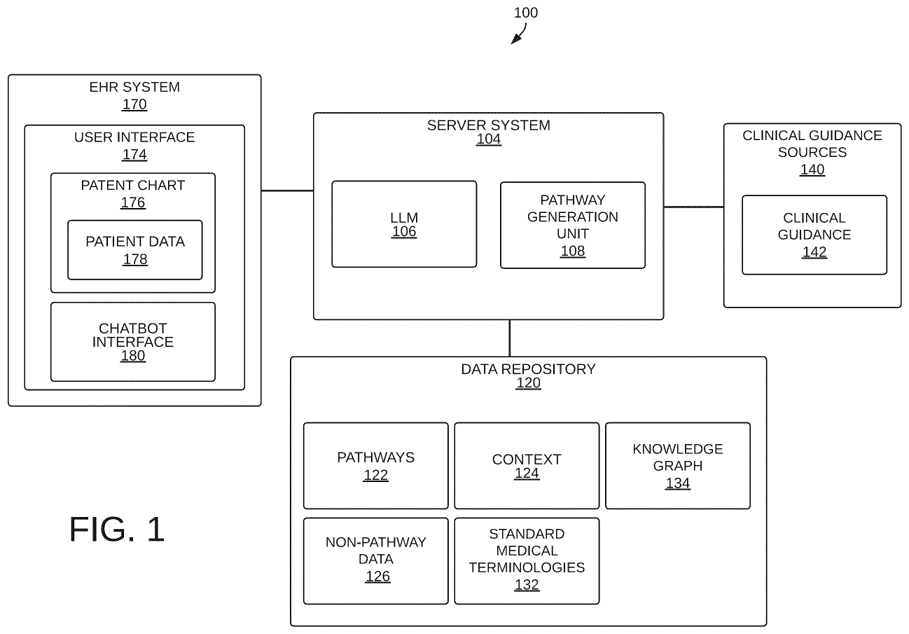

FIG. 1 illustrates system 100 for augmenting EHR patient charts in accordance with one or more embodiments. System 100 generates a pathway from clinical guidance using an LLM, where the generated pathway is a structured summary for a specific disease state, produces a patient chart in an EHR system with information derived from the pathway.

As illustrated in FIG. 1, information management architecture system 100 comprises server system 104, LLM 106, pathway generation unit 108, data repository 120, pathways 122, context 124, non-pathway data 126, standard medical terminologies 132, knowledge graph 134, clinical guidance sources 140, clinical guidance 142, EHR system 170, user interface 174, patient data 178, and chatbot interface 180. In one or more embodiments, system 100 may include more components or fewer components than the components illustrated in FIG. 1.

In an embodiment, EHR system 170 serves as the central hub for managing patient health records in a healthcare environment. EHR system 170 stores and organizes patient information, including detailed medical histories, current and past diagnoses, prescribed medications, treatment plans, and outcomes of previous interventions. EHR system 170 supports interoperability standards, enabling seamless data exchange with other healthcare systems and devices.

In an embodiment, user interface 174 is the gateway through which healthcare professionals interact with patient data. User interface 174 efficiently organizes, displays, and facilitates the input of medical information. User interface 174 typically includes various features, such as patient dashboards, data entry forms, search functions, and alert systems. User interface 174 enables doctors and nurses to easily access and update patient records, order tests, review results, and documents.

In an embodiment, patient chart 176 within EHR system 170 organizes and displays patient-specific information in a comprehensive and easily navigable format. patient chart 176 serves as a centralized hub for relevant patient data, including medical history, current medications, allergies, lab results, and treatment plans. Patient chart 176 presents patient information in a logical, hierarchical structure, allowing healthcare providers to quickly access and update specific sections as needed. Interactive elements, such as tabs, dropdown menus, and clickable icons, facilitate efficient navigation through the various components of the patient's record, ensuring that critical information is readily available to support informed clinical decision-making.

In an embodiment, patient data 178 encompasses relevant medical information for individual patients, forming a comprehensive digital health record. Patient data 178 includes extensive medical information, such as a comprehensive medical history, including past diagnoses, surgeries, and treatments, current and past medications, including dosages and adverse reactions, allergies and sensitivities, immunization records, lab and imaging results, vital signs and other biometric data, treatment plans and care goals, and notes from healthcare provider encounters.

In an embodiment, chatbot interface 180 provides an AI-driven interface within the EHR system 170. Chatbot interface 180 allows healthcare professionals to interact with the system using natural language queries and commands. Chatbot interface 180 is an interface for numerous tasks, such as retrieving specific patient information, scheduling appointments, ordering tests, and providing basic clinical decision support. Chatbot interface 180 uses natural language processing (NLP) to interpret user inputs and interacts with machine learning (ML) systems to provide responses. Chatbot interface 180 interacts with server system 104 to obtain information from LLM 106, pathway 122, and knowledge graph 134.

In an embodiment, clinical guidance source 140 is a source of clinical guidance 142. In one example, clinical guidance source 140 is a website or other source of clinical guidance 142. This source serves as a comprehensive repository of up-to-date medical information, best practices, and treatment protocols. In one example, clinical guidance source 140 includes evidence-based guidelines, drug interaction databases, diagnostic criteria, and treatment algorithms.

In an embodiment, clinical guidance 142 is text-based information that includes specific medical advice and treatment protocols. Clinical practice guidelines are systematically developed statements to assist practitioner and patient decisions about appropriate health care for specific clinical circumstances. Clinical guidance 142 is typically issued by third-party organizations and defines the role of specific diagnostic and treatment modalities in the diagnosis and management of patients. The statements include recommendations that are based on evidence from a rigorous systematic review and synthesis of the published medical literature. Clinical guidance 142 typically incorporates guideline recommendations based on real-world evidence and outcome data. Clinical guidance 142 is a critical reference point for healthcare professionals, offering standardized, evidence-based approaches to patient care. Exemplary, clinical guidance 142 covers a wide range of medical scenarios, from common ailments to complex chronic conditions. Clinical guidance 142 includes various information, such as dosage recommendations for medications, potential drug interactions and contraindications, and references to relevant clinical studies.

In an embodiment, server system 104 analyses clinical guidance 142 and produces pathway 122 using LLM 106 and pathway generation unit 108. In an embodiment, pathway 122 is a structured clinical workflow for specific medical conditions that provides a standardized approach to patient care. Pathway 122 incorporates decision points and branching logic to accommodate various patient scenarios and comorbidities. EHR system 170 uses pathway 122 to provide clinical decision support with real-time guidance to healthcare providers at the point of care.

In an embodiment, pathway generation unit 108 creates pathway 122 using LLM 106. In an embodiment, LLM 106 is a type of artificial intelligence (AI) system designed to understand, generate, and manipulate human language on a large scale. These models are trained on vast amounts of text data, allowing them to recognize patterns, context, and nuances in language. LLMs perform a wide range of tasks, including text generation, translation, summarization, question-answering, and even basic reasoning. They use complex neural network architectures, typically based on transformer models, to process and generate text. In one example, LLM 106 undergoes training and fine-tuning using domain-specific medical corpora to improve its understanding of specialized medical terminology and concepts. In one example, LLM 106 incorporates attention mechanisms and transformer architectures to capture long-range dependencies in medical texts.

In an embodiment, LLM 106 generates pathway 122 using context 124 and clinical guidance 142. Context 124 acts as framework to aid in the generation of pathway 122 from clinical guidance 142. In one example, context 124 includes formatting and content rules for pathways that aid LLM 106 in constructing the pathway 122.

In an embodiment, LLM 106 also generates non-pathway data 126 that encompasses medical information not directly related to clinical pathways. Server system 104 employs text mining and NLP techniques to extract relevant information from clinical guidance 142 and other sources. EHR system 170 uses non-pathway data 126 to improve information provided to the medical provider.

In an embodiment, standard medical terminologies 132 ensure consistent and standardized representation of medical concepts, facilitating seamless data exchange and interoperability. Exemplary standard medical terminologies 132 include SNOMED, ICD-10, RxNorm, and LOINC. LLM 106 codifies concepts in pathway 122 and knowledge graph 134 using standard medical terminologies 132. In one example, server system 104 classifies these concepts and constructs a knowledge graph 134.

In an embodiment, knowledge graph 134 represents complex relationships between medical concepts and entities, providing a rich semantic network for advanced clinical reasoning. Knowledge graph 134 utilizes ontology-based modeling to capture hierarchical and associative relationships between medical concepts. Server system 104 compares patient data against knowledge graph 134. Based on the comparison, server system 104 generates suggestions and interventions for healthcare providers through EHR system 170.

In one or more embodiments, data repository 120 is any type of storage unit and/or device (e.g., a file system, database, collection of tables, or any other storage mechanism) for storing data. Furthermore, data repository 120 may include multiple different storage units and/or devices. The multiple different storage units and/or devices may or may not be of the same type or located at the same physical site. Furthermore, data repository 120 may be implemented or executed on the same computing system as server system 104. Additionally, or alternatively, data repository 120 may be implemented or executed on a computing system separate from server system 104. Data repository 120 may be communicatively coupled to server system 104 via a direct connection or via a network.

In one or more embodiments, server system 104, LLM 106, pathway generation unit 108, data repository 120, clinical guidance source(s) 140, EHR system 170, user interface 174, patient data 178, and chatbot interface 180 refer to hardware and/or software configured to perform operations described herein for information storage and retrieval.

In an embodiment, server system 104, LLM 106, pathway generation unit 108, data repository 120, clinical guidance source(s) 140, EHR system 170, user interface 174, patient data 178, and chatbot interface 180 are implemented and/or stored on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

### 3. AUGMENTING ELECTRONIC HEALTH RECORD PATIENT CHARTS

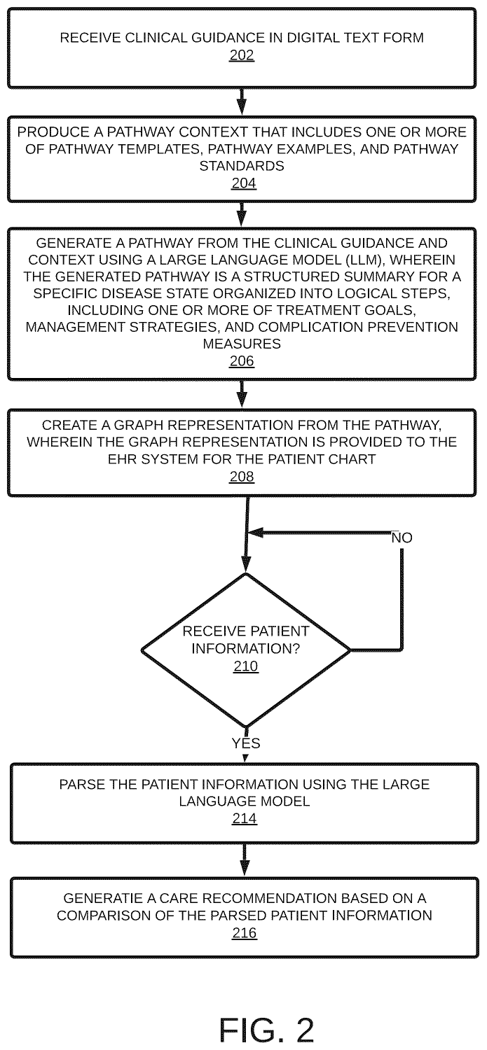

FIG. 2 illustrates an example set of operations for augmenting EHR patient charts in accordance with one or more embodiments. One or more operations illustrated in FIG. 2 may be modified, rearranged, or omitted. Accordingly, the particular sequence of operations illustrated in FIG. 2 should not be construed as limiting the scope of one or more embodiments.

The system takes clinical guidance, that exists as text, and employs large language models (LLMs) to identify pathways. These pathways are then extracted, codified, and classified. A knowledge graph is created from the extracted information. The system addresses the challenge of lengthy, verbose guidance documents that require conversion into actionable rules for clinical systems. The current process of rule creation by developers is slow, typically taking months of communication and development. The resulting rules are difficult for clinicians to read and validate.

The system aims to overcome limitations in the current approach. During the translation process, many clinical features are often lost. The system proposes complete integration and faster market delivery. The LLM-based approach helps close gaps that might exist with traditional rule-based systems. Clinical guidance changes frequently, sometimes every 6 months, and understanding these changes takes considerable time to implement and publish. The system's use of LLMs and graphs enables more effective adaptation to these changes.

In an embodiment, a system receives clinical guidance in digital text form (Operation 202). Clinical guidance is evidence-based recommendations and best practices developed to assist healthcare professionals in making decisions about appropriate patient care for specific clinical circumstances. Clinical guidance is human readable but is often verbose and difficult to use due to time constraints and heavy workload of the healthcare providers. As discussed below, the system processes the received clinical guidance to extract relevant information for patient care. An LLM analyzes the text to identify key elements and structures within the guidance.

In an embodiment, the system produces a pathway context that includes pathway templates, pathway examples, and pathway standards (Operation 204). The pathway context serves as a framework for organizing and interpreting the clinical guidance by an LLM. The system uses the pathway context to guide the generation of structured pathways from the received guidance. The context is provided to the LLM to produce pathways from clinical guidance. The context serves as a specialized framework that configures the model for this specific task. The context encompasses a clear definition of the objective, transforming narrative clinical guidelines into structured, step-by-step pathways. The context provides the necessary parameters to achieve this goal. The context primes the LLM with relevant medical terminology and concepts while specifying the desired format and essential elements of the output pathways. In one example, the context includes interpretation rules for handling ambiguities, quality assurance criteria to ensure clinical validity and practical applicability, and ethical guidelines emphasizing patient safety. The context effectively transforms the LLM into a specialized tool for clinical pathway development, balancing deep medical knowledge with practical healthcare implementation needs.

In an embodiment, the system generates a pathway from the clinical guidance and context using an LLM (Operation 206). The generated pathway represents a structured summary for a disease state. The pathway organizes information into logical steps, encompassing treatment goals, management strategies, and complication prevention measures. The LLM ensures the pathway accurately reflects the content and intent of the original clinical guidance.

In an embodiment, the system creates or updates a knowledge graph representation from the generated pathway (Operation 208). The knowledge graph depicts the relationships between different elements of the pathway. The system provides the knowledge graph to an EHR system for inclusion in patient charts. The graph representation enhances the usability and interpretability of the pathway information within the EHR.

In an embodiment, the system checks for the receipt of patient information (Operation 210). The system continually monitors for incoming patient data from various sources. When patient information becomes available, the system proceeds to process and analyze the data. If the patient information is received, the system proceeds to operation 214. If the patient information is not received, the system repeats operation 210.

In an embodiment, the system parses the received patient information using the LLM (Operation 214). The LLM extracts relevant clinical concepts and details from the patient information. The parsing process structures the patient data for further analysis and comparison with the generated pathways.

In an embodiment, the system generates a care recommendation based on a comparison of the parsed patient information (Operation 216). The system evaluates the parsed patient data against the previously generated pathways and graph representations. The care recommendation incorporates insights from the clinical guidance while considering the specific patient context. The system provides tailored suggestions for patient care based on the analysis.

In an embodiment, the system parses patient information using LLMs, codifying the data to determine the patient's location within a care pathway. With this comprehensive information, the system provides functionality, such as suggesting alternative medications, optimizing treatment options, or validating if a diagnosis has sufficient clinical features for support.

In an embodiment, the system analyzes patient charts and extracts concepts using LLMs. Concepts are codified as much as possible using standard terminologies like Systematized Nomenclature of Medicine Clinical Terms (SNOMED CT), International Classification of Diseases, 10th Revision (ICD-10), RxNorm, and Logical Observation Identifiers Names and Codes (LOINC). The system classifies these concepts and constructs a graph representation. This graph serves as a golden standard against which patient data is compared. Based on the comparison, the system generates suggestions and interventions for healthcare providers.

In an embodiment, the system considers contextual information when providing recommendations. For instance, if a patient is pregnant and has diabetes but is being seen by an obstetrician, the system focuses on gaps in pregnancy-related care rather than diabetes management. The system triggers appropriate pathways based on listed problems or diagnoses such as acute kidney failure.

In an embodiment, to address potential hallucinations in LLM outputs, the system implements several strategies. For guidelines data, human oversight is incorporated into the process. For individual patient data, where constant human monitoring is impractical, automatic processes are implemented to reduce the chance of hallucinations. The system employs cross-referencing validation that compares LLM-generated medication recommendations against pharmaceutical databases to verify drug existence and correct spelling. The system performs dosage range verification by checking recommended doses against established prescribing information databases and clinical dosing calculators to identify potentially dangerous recommendations that exceed safe therapeutic ranges. The system distinguishes between information generated by LLMs and information vetted by humans, presenting this distinction in the user interface.

In an embodiment, for suggesting alternate medications, the system considers the patient's current condition, diagnosis, and known allergies. When a provider asks for alternative medications due to patient allergies or side effects, the system pulls relevant pathways or guidelines and finds appropriate options. The system improves treatment options based on various factors, such as cost or side effects, providing alternatives that may have fewer adverse effects within the scope of the clinical guidance.

In one embodiment, for differential diagnosis, the system expands on preliminary working diagnoses. When a patient presents with a high-level problem, such as acute abdominal pain, the system suggests potential diagnoses, like pancreatitis or appendicitis. The system recommends additional checks or tests to help providers differentiate between possible diagnoses, assisting in the systematic elimination process to determine the patient's exact condition.

In an embodiment, the system ensures critical features for diagnoses are captured to support proper coding and avoid missing important information. By analyzing the patient chart and comparing it to the established guidelines, the system identifies any missing elements crucial for accurate diagnosis and proper reimbursement. The system alerts providers to potential gaps in documentation before the patient encounter concludes, eliminating the need for time-consuming back-and-forth communications between coding staff and healthcare providers.

In an embodiment, to address care gaps, particularly in opioid management, the system integrates information from various sources. Instead of relying on separate dashboards and administrative reports, the system makes information actionable within the clinical workflow. For instance, the system prompts providers to check the Prescription Drug Monitoring Program (PDMP) before prescribing opioids for more than three days, aligning with clinical guidelines and quality measures. Algorithms analyze this integrated data to identify potential opioid-related risks and care gaps in real time. The system incorporates ML models trained on historical prescribing patterns and patient outcomes to predict high-risk scenarios.

In an embodiment, within the clinical workflow, the system generates context-aware prompts and alerts. For example, when a provider initiates an opioid prescription, the system automatically checks the duration. If the prescription exceeds three days, the system triggers a prompt for the provider to check the PDMP. This prompt includes a direct link to the PDMP interface, streamlining the verification process. The system also presents a summary of the patient's opioid prescription history, including prescriptions from other providers, to offer a comprehensive view of the patient's opioid use.

In an embodiment, the system extends beyond simple reminders by offering actionable recommendations. It suggests alternative pain management strategies based on the patient's medical history, current medications, and evidence-based guidelines. These recommendations consider different factors, such as the patient's risk for opioid use disorder, potential drug interactions, and comorbidities. The system also tracks quality measures related to opioid prescribing in real time, allowing providers to monitor their adherence to best practices and regulatory requirements.

In one embodiment, the system's immunization forecasting capability validates existing vaccine doses and suggests missing vaccinations based on standard schedules or specific requirements. The system considers numerous factors, such as the patient's travel plans, occupation, or age, to provide tailored immunization recommendations. The system evaluates the validity of administered doses, accounting for factors like incorrect intervals or suboptimal dosing, and suggests additional doses or catch-up schedules as needed.

In an embodiment, the system validates vaccine doses and determines missing vaccinations according to standard immunization schedules through comprehensive forecasting operations. The system processes patient vaccination history including dose administration dates, and interval timing between doses to assess validity according to clinical guidelines. The system evaluates factors such as patient age at vaccination, minimum intervals between doses, and maximum age limits for specific vaccines to determine dose effectiveness. The system considers patient travel plans, occupation requirements, and age-specific recommendations to generate tailored immunization forecasts. The system accounts for special populations including preterm infants weighing less than two kilograms and infants born to hepatitis B surface antigen positive mothers who require modified vaccination schedules. The system suggests catch-up schedules when dose intervals were incorrect or suboptimal, recommending additional doses based on current clinical guidelines from Centers for Disease Control immunization schedules.

In an embodiment, the system transforms verbose clinical guidance documents into structured, actionable pathways. LLMs analyze the guidance and produce a structured summary, or pathway, for specific disease states. The pathway organizes information into logical steps, grouping activities and decision points. For example, a diabetes management pathway includes sections on treatment goals, specific management strategies, and complication prevention.

In an embodiment, the system codifies concepts within the pathway using standard medical terminologies. Concepts without specific codes are still recognized and incorporated into the decision-making process. The resulting graph representation allows for a more holistic view of patient care, moving beyond step-by-step workflows to consider relevant factors simultaneously.

In an embodiment, the system combines information from multiple sources to create enriched pathways. Clinical guidelines, existing rules, ML models, and dashboard data are integrated to form a more complete picture of patient care requirements. This comprehensive approach allows the system to address both clinical and administrative aspects of care, such as quality measures and regulatory reporting.

In an embodiment, the system applies LLMs to bridge gaps between codified information and unstructured text in clinical notes. When certain concepts lack specific codes but remain important for decision making, the system utilizes LLMs to interpret and incorporate this information into the care recommendations. This feature allows for more nuanced and context-aware suggestions that go beyond rigid, code-based rules.

In an embodiment, the system provides an interface for clinicians to interact with the AI-generated recommendations. Suggestions are presented with links to relevant sections of the patient chart or clinical guidelines, allowing providers to quickly verify the basis for recommendations. The system distinguishes between information derived from established guidelines and insights generated by LLMs, enabling clinicians to make informed decisions about following the suggestions.

In an embodiment, the system incorporates a quality control mechanism to validate and improve its recommendations. By comparing the LLM-generated pathways with existing rule-based systems and ML models, the system identifies potential gaps or inconsistencies in current clinical decision support tools. This comparison helps ensure comprehensive coverage of clinical scenarios and enhances the overall quality of care recommendations.

In an embodiment, the system adapts to complex patient scenarios involving multiple conditions. For patients with comorbidities, such as diabetes and pregnancy, the system integrates relevant pathways to provide holistic care recommendations. The graph-based representation allows for seamless combination of guidance from different clinical domains, ensuring that different aspects of a patient's health are considered in the care plan.

In an embodiment, the system supports continuous improvement of clinical content. As new medical knowledge becomes available, the system rapidly incorporates updates into existing pathways. This dynamic approach ensures that care recommendations remain current with the latest clinical evidence and best practices. The system's ability to quickly process and integrate new information addresses the challenge of keeping clinical decision support tools up-to-date in a rapidly evolving healthcare landscape.

In an embodiment, the system enhances the efficiency of clinical documentation and coding. By analyzing patient encounters in real time, the system identifies potential documentation gaps that could affect coding accuracy and reimbursement. The system prompts clinicians to address these gaps during the patient visit, reducing the need for retrospective queries and documentation updates. This proactive approach improves the accuracy of clinical records and streamlines the billing process.

In an embodiment, the system facilitates more personalized care recommendations by considering a range of patient-specific factors. Beyond basic demographic information and diagnoses, the system incorporates data on patient preferences, social determinants of health, and previous treatment responses. The system analyzes patient preference data collected from previous clinical encounters by parsing electronic health record notes for documented communication preferences, treatment modality selections, and appointment timing requirements using natural language processing algorithms. The system evaluates previous treatment responses by analyzing medication adherence patterns from prescription refill histories, documented adverse drug reactions from allergy profiles, and treatment outcome measurements from laboratory result trends and clinical assessment scores. This comprehensive approach enables the generation of highly tailored care plans that align with individual patient needs and circumstances.

In an embodiment, the system incorporates a feedback loop mechanism to continuously refine its recommendations. Clinician interactions with the system, including acceptance or rejection of suggestions, are logged and analyzed. This data informs future updates to the system's algorithms, improving the relevance and accuracy of recommendations over time.

In an embodiment, the system employs NLP capabilities to extract relevant information from unstructured clinical notes in patient charts. By analyzing text entries, the system identifies key clinical concepts and incorporates them into the patient's care graph. This feature ensures that important details documented in narrative form are not overlooked when generating care recommendations.

### 4. DIABETES MANAGEMENT PATHWAY GENERATION EXAMPLE

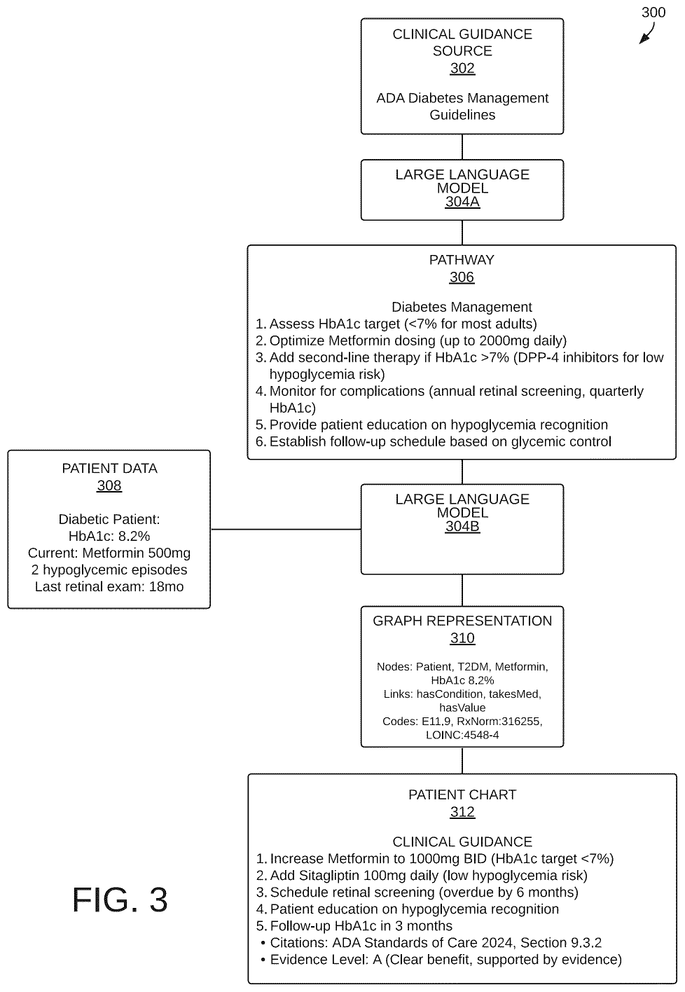

FIG. 3 illustrates an example of generating structured care recommendations by processing diabetes management clinical guidance. One or more operations or elements illustrated in FIG. 3 may be modified, rearranged, or omitted. Accordingly, the particular sequence of operations and elements illustrated in FIG. 3 should not be construed as limiting the scope of one or more embodiments.

In an embodiment, the system transforms clinical guidelines into patient-specific care recommendations through automated processing operations. The system processes clinical guidelines for multiple medical domains, such as diabetes, cardiology, oncology, and pulmonology. Diabetes care serves as a representative example demonstrating transformation of narrative guidelines into executable clinical workflows. The system transforms unstructured text through NLP to generate structured recommendations with specific medication dosages, monitoring intervals, and intervention thresholds. Healthcare providers receive outputs formatted for direct clinical implementation across diverse medical specialties.

In an embodiment, clinical guidance source 302 stores evidence-based treatment protocols in text format. Source 302 incorporates clinical documentation from professional medical organizations, including diagnostic criteria, treatment targets with numerical thresholds, medication algorithms with dosing ranges, monitoring schedules, and patient education specifications.

In an embodiment, LLMs 304A and 304B utilize transformer-based neural network architectures to process text data. Models employ multi-layer attention mechanisms that analyze relationships between words and phrases across variable context windows, ranging from hundreds to thousands of tokens. Self-attention layers compute weighted relationships between input elements, enabling identification of clinical concepts separated by intervening text. Models incorporate pre-trained weights derived from medical literature corpora, enabling recognition of specialized terminology and clinical relationships. Fine-tuning processes adapt general language understanding to medical domain requirements through exposure to annotated clinical guidelines and treatment protocols. Models generate structured outputs by mapping identified clinical concepts to predefined schema elements, converting free-text descriptions into formatted data structures.

In an embodiment, LLM 304A processes textual content from source 302 to identify treatment protocols, numerical targets, and procedural sequences. LLM 304A transforms narrative descriptions into pathway 306 using pattern recognition and semantic analysis. LLM 304A generates discrete management steps linked by conditional logic and quantified parameters.

In an embodiment, pathway 306 organizes clinical management into sequential decision trees. For diabetes management, pathway 306 specifies glycated hemoglobin targets of less than 7 percent for adult patients, medication titration protocols for Metformin up to 2000 milligrams daily, second-line therapy criteria triggered when glycated hemoglobin exceeds 7 percent, complication screening intervals that include annual retinal examinations and quarterly laboratory testing, patient education modules addressing hypoglycemia symptoms, and follow-up timing based on glycemic control measurements. Pathway 306 provides template structure adaptable across medical specialties through parameter modification.

In an embodiment, patient data 308 supplies clinical measurements for an individual patient assessment. Sample parameters include glycated hemoglobin measurement of 8.2 percent, current Metformin prescription of 500 milligrams daily, documented hypoglycemic episode count of 2 events, and retinal examination date 18 months prior to current evaluation. Patient data 308 enables automated comparison against pathway 306 thresholds to trigger appropriate interventions.

In an embodiment, LLM 304B combines patient data 308 with pathway 306 to construct graph representation 310. Graph representation 310 structures clinical information as interconnected nodes and relationships. Nodes represent clinical entities, including patient identifier, Type 2 Diabetes Mellitus diagnosis, Metformin medication entity, and glycated hemoglobin value of 8.2 percent. Relationships connect nodes through semantic links, including hasCondition, takesMedication, and hasLabValue predicates. Graph representation 310 incorporates standardized medical coding including International Classification of Diseases code E11.9 for Type 2 Diabetes Mellitus, RxNorm identifier 316255 for Metformin, and Logical Observation Identifiers Names and Codes identifier 4548-4 for glycated hemoglobin measurements. Standardized coding enables interoperability across healthcare information systems.

In an embodiment, system generates patient chart 312 from graph representation 310 through rule-based inference. Patient chart 312 presents actionable recommendations formatted for clinical workflow integration. Recommendations specify Metformin dosage increase to 1000 milligrams twice daily targeting glycated hemoglobin below 7 percent, Sitagliptin initiation at 100 milligrams daily selected for reduced hypoglycemia risk profile, retinal screening appointment scheduling to address 6-month overdue status, patient education session focusing on hypoglycemia symptom recognition, and glycated hemoglobin retesting scheduled within 3-month interval. Patient chart 312 demonstrates automated generation of personalized treatment plans aligned with evidence-based guidelines.

In an embodiment, the system processes diabetes management pathway outputs from LLM 304B to generate clinical interface elements that transform structured recommendations into actionable healthcare provider workflows. The system receives pathway 306 including diabetes management protocols including glycated hemoglobin targets of less than seven percent for adult patients and Metformin titration protocols ranging from five hundred to two thousand milligrams daily, automatically rendering medication adjustment interfaces with dosage selection controls and frequency options. The system processes patient data 308 showing glycated hemoglobin measurement of eight point two percent and current Metformin prescription of five hundred milligrams daily to generate alert notifications displaying the patient's above-target glucose control status with recommended medication increases. The system transforms graph representation 310 including nodes for patient identifier, Type Two Diabetes Mellitus diagnosis, Metformin medication entity, and glycated hemoglobin value into interactive patient summary dashboards showing medication timelines, laboratory result trends, and care milestone tracking with detailed information access. The system processes pathway recommendations for Metformin dosage increase to one thousand milligrams twice daily and Sitagliptin initiation at one hundred milligrams daily to generate prescription order forms with hypoglycemia risk assessment and drug interaction screening. The system creates patient chart 312 interface elements including clinical note templates with assessment sections describing current glucose control status, treatment plan modifications, and follow-up care requirements that integrate with electronic health record systems for provider workflow implementation.

In an embodiment, the system processes LLM outputs to generate dynamic user interface elements through real-time rendering algorithms that transform textual recommendations into interactive clinical workflow components. The system receives LLM-generated medication suggestions and automatically renders prescription entry forms with pre-populated drug names, dosage ranges, and frequency selections derived from clinical pathway analysis. The system creates interactive medication reconciliation displays by processing LLM comparisons between prescribed medications and patient-reported drug lists, generating visual indicators that highlight discrepancies, potential duplications, and missing medications. The system transforms LLM-generated care timeline recommendations into graphical calendar interfaces showing treatment milestones, appointment schedules, laboratory monitoring requirements, and follow-up care organized chronologically with clickable event details. The system processes LLM analysis of patient risk factors to render dashboard widgets displaying numerical risk scores, trend graphs, and alert notifications that update automatically when new patient data becomes available. The system generates interactive care plan flowcharts by converting LLM pathway recommendations into clickable decision trees with branching logic that guides healthcare providers through treatment protocols based on patient-specific conditions and responses.

In an embodiment, the system executes automated technical operations by processing LLM outputs into structured database updates, healthcare application functions, and/or clinical decision support mechanisms that integrate across multiple medical software systems. The system converts LLM-generated clinical assessments into database records including care plan tracking updates, prescription history modifications, patient risk stratification scores, and quality metric calculations that trigger automated care management protocols and provider performance reporting. The system processes LLM recommendations to execute healthcare application functions including prior authorization form population, immunization reminder generation, specialist referral creation, laboratory order set automation, and patient education material distribution based on literacy levels and health conditions. The system transforms LLM analysis into real-time clinical decision support through automated pharmacy alerts during prescription entry, care gap notifications with specific action items, differential diagnosis tools with ranked possibilities, quality measure compliance tracking, and population health management protocols that identify high-risk patient cohorts for targeted intervention campaigns.

### 5. ADDITIONAL INFORMATION

In an embodiment, a reference enterprise data model establishes cross-enterprise processes for governance, cataloging entity assets, validation, enforcement, and team review activities. The reference enterprise data model delivers structured phases with defined deliverables and measurable values while identifying delegates from participating teams. Initial phases involve cataloging existing entities and attributes for individual domains, followed by creation of base reference enterprise data model structures that hold entities and attributes with semantic understanding mapped to data standards.

In an embodiment, the reference enterprise data model provides pre-coordinated and post-coordinated concepts and expressions that determine interoperability levels and semantic commonalities. The reference enterprise data model enables semantic explanation capabilities, identifies interoperability patterns and integration patterns, and supports automated building and testing processes. Base structures for business subdomain entity data models encompass finance with data standards, clinical data with standards, reporting analytics, reporting functions, data warehousing operations, and compliance requirements.

In an embodiment, enterprise reference entity data model use cases span global health care EHR systems, such as Siemens MedSuite or Oracle Health EHR (formerly Cerner Millennium), for clinicals, financials, laboratory and laboratory quality control, and pharmacy operations, such as decision support systems and clinical conflict checking. Clinical documentation systems, such as Soarian Clinicals, MedSuite, and ISHMeds, integrate with reference enterprise data model through standardized interfaces.

In an embodiment, integration platforms facilitate connections between multiple proprietary EHR systems through integration architectures. Interoperability functions enable reconciliation between health information systems and EHR systems for problems, immunizations, and medications using HL7 and NCPDP workflows with ISO standards compliance. Electronic health record and financial systems connect with real-world data sources for pharmaceutical companies and regulatory agencies.

In an embodiment, a knowledge management system implements workflow processes for creating new integrated local dictionary structures. The knowledge management system selects new master dictionaries for medications, problems, laboratories, and related medical concepts, then creates and customizes local dictionaries tethered to master dictionary sources. Publication processes integrate local dictionaries to local systems while following maintenance workflows for integrated local dictionary management.

In an embodiment, the knowledge management system integrates existing local dictionary workflows by importing local dictionaries from EHR systems for medications, problems, laboratories, and related concepts. Tethering processes connect local dictionaries to corresponding master dictionary sources, followed by reconciliation of local items with tethered master dictionary content. Publication processes deliver integrated local dictionaries to local systems with subsequent maintenance workflow implementation.

In an embodiment, maintenance workflows for integrated local dictionaries involve re-importing local dictionaries with updates, reconciling updates with tethered master dictionary sources, and publishing integrated local dictionaries to an EHR system. Notification systems receive master dictionary updates and trigger appropriate reconciliation processes to maintain synchronization between local and master dictionary sources.

In an embodiment, AI workflow processes receive notifications from AI services regarding proposed classes for master dictionaries. A knowledge management system reviews proposed classes, reconciles updates with tethered master dictionary sources, and publishes updates to runtime systems. Notification systems enable automated proposal generation while maintaining human oversight for validation and approval processes.

In an embodiment, an ontology conversion system leverages content from existing ontology structures and converts ontological representations to logical formalism frameworks.

Conversion processes address combinatorial explosion challenges and safety requirements while providing background knowledge for new axiom identification. Analysis encompasses atrial fibrillation care process models, standard atrial fibrillation protocols, condition management, immunization tracking, medication management, sepsis treatment protocols, and clinical decision support systems.

In an embodiment, a core concept model transforms ontology concepts from term-based representations to context-validated concepts. The core concept model establishes relationships beyond simple member relationships, incorporating hierarchical descriptions as ontological concepts. Concept structures include associated code sets with defined vocabularies, contexts, concept classes, and group memberships with subset relationships.

In an embodiment, the seamless exchange system addresses continuous client modifications to coded values without visibility into downstream impacts. Modified diagnosis codes potentially disrupt clinical decision support reminders or Fast Healthcare Interoperability Resources FHIR) mapping processes. Knowledge-based terminology service prototypes provide solutions for faster implementation through federated maintenance of concept relationships, elimination of knowledge asset silos, and reduced labor requirements for knowledge creation.

In an embodiment, a knowledge-based terminology service enables smart queries, meaningful data leverage with connections and representation, creation of computable artifacts, and galvanized decision support capabilities. Data quality improvements support patient care and research activities while reducing free text usage through better concept matching for codable concepts lacking coding or mapping relationships.

In an embodiment, a clinical decision support system infers and augments information within patient charts to supplement data not directly available in chart records. Semantic knowledge creation provides decision support through systematic analysis of patient medication lists, problem lists, allergies, and laboratory results.

In an embodiment, a clinical decision support system suggests alternative medications, optimizes treatment options, suggests differential diagnoses, validates clinical features of diagnoses, identifies treatment care gaps including opioid-related gaps, and translates clinical guidance and pathways into actionable clinical interventions for various conditions, such as diabetes mellitus and acute kidney failure. Immunization forecasting capabilities integrate with decision support functions.

In an embodiment, a digital assistant system for healthcare providers is a technology platform designed to streamline clinical workflows and administrative tasks through automation, voice recognition, and AI-powered tools. The digital assistant system helps healthcare professionals work more efficiently while maintaining focus on patient care. The digital assistant system for healthcare providers represents technological advancement in healthcare technology that enhances provider experiences and improves patient care outcomes. The digital assistant system focuses on connections between problems, diagnoses, medications, immunizations, procedures, and laboratory results to ensure providers access necessary information for clinical decision-making.

In an embodiment, the medication ontology establishes relationships between pharmaceutical classes, medications, conditions, contraindications, active ingredients, diagnoses, and anatomical structures. Medication ontology incorporates drug information from RxNorm, Multum, SNOMED CT, and ICD-10 sources with common side effects, post-marketing reported side effects, and contraindication relationships.

In an embodiment, the immunization ontology encompasses viral vaccines, drug components, contraindications, allergies, conditions, and severity factors. The immunization ontology processes patient age, gender, National Drug Code identifiers, Current Procedural Terminology codes, and ICD-10 diagnosis codes to determine vaccine administration appropriateness and contraindication identification.

In an embodiment, the immunization ontology evaluates specific vaccine scenarios, such as rotavirus and hepatitis B vaccine administration, for patients with severe combined immunodeficiency disease, vaccine component allergies, and previous vaccination history. Ontological relationships enable dynamic inference of contraindications based on patient-specific factors and vaccine composition data.

In an embodiment, the technical implementation framework incorporates runtime storage systems, smart query capabilities, reasoning engines for design and runtime operations, full-text search functionality, and numeric search with computed search capabilities.

In an embodiment, a concept categorization system enables knowledge architects and clients to add and integrate new terminology concepts, value sets, categories, and terminology systems. New variant identification encompasses emerging conditions, such as COVID variants, and experimental treatments through systematic evaluation processes. Code set standardization decisions span enterprise-wide implementations through comparative analysis of terminology definitions across organizational systems.

In an embodiment, the concept categorization system identifies unmatched codes, redundant codes, and orphaned codes across content management tools, forms, workflows, and external sources. Client code set integration drives standards updates through collaborative feedback mechanisms. Platform synchronization capabilities enable content integration across Event Set Hierarchy, Bedrock, Content Management Tools, and Ontology tools.

In an embodiment, development teams categorize concepts to enable smarter deduplication processes and improved ranking algorithms. Contextual data linking, retrieval, and display capabilities enhance concept understanding. Patient record curation functions support provider decision-making through recommendation engines and smart search capabilities.

In an embodiment, a provider management system addresses complex patient scenarios where conditions span multiple medical specialties. Nephrotic syndrome caused by diabetes type conditions appear on problem lists for nephrology and endocrinology specialists through shared diagnostic categorization. Competitive advantages emerge through interoperable platforms and knowledge-driven applications for managing complex patient populations.

In an embodiment, a seamless exchange reconciliation enables clinician users to view concepts grouped by parent classifications, including food allergies, beta blockers, and anticoagulants. Problem groupings encompass high cholesterol with hyperlipidemia, diabetes with diabetes mellitus, and related condition clustering. Patient summary document generation utilizes categorization functions for organized information presentation.

In an embodiment, a clinical guidance documentation system processes clinical pathways through LLM extraction and codification processes. Concept classification creates knowledge graphs from clinical documentation, while patient data extraction and codification generates subgraph structures for individual patients. Graph-based representations enable attribute application without complete modeling requirements.

In an embodiment, an LLM processing creates relationships, performs extraction operations, and interprets usage patterns from clinical guidance data. Machine learning methods target recommendation use cases, including missing vaccination identification, treatment optimization, and care gap analysis. Codification processes transform unstructured clinical guidance into computable formats suitable for automated decision support.

In an embodiment, clinical decision rules for immunization forecasting translate medical guidelines into logical statements. Hepatitis B recommended ranges encompass birth to four weeks for initial doses, four weeks to sixteen weeks for second doses, and six months to nineteen months for final doses. Special population considerations include preterm infants and infants born to hepatitis B surface antigen positive mothers.

In an embodiment, a diabetes mellitus management system integrates with chronic kidney disease risk assessment through standardized clinical guidelines. American Diabetes Association Standards of Care provide foundational frameworks for diabetes care, general treatment goals, and quality evaluation tools. Professional Practice Committee oversight ensures annual updates and evidence-grading systems for clinical practice recommendations.

In an embodiment, drug indication analysis connects Sotalol with SNOMED CT concepts through hasMultumIndication relationships. Subclass relationships link Sotalol classifications to broader pharmaceutical categories while maintaining specific indication mappings. Query capabilities enable traversal from drug concepts to condition hierarchies, side effect profiles, and contraindication relationships. A silver sulfadiazine drug example demonstrates side effect relationship modeling through hasMultumCommonSideEffect properties. Common side effects include leukopenia, application site rash, and pruritus conditions with corresponding SNOMED CT mappings. Query functionality retrieves drugs with specific side effect profiles and conditions treated by particular medications.

In an embodiment, a drug interaction visualization represents complex relationship networks between pharmaceutical substances and medical conditions through graph-based representations. Visual mapping displays connections between drug classifications, therapeutic indications, contraindications, and patient-specific factors. Interactive exploration enables clinicians to understand medication impacts across multiple physiological systems.

In an embodiment, medication ontology insights incorporate pharmaceutical classes, medication relationships, contraindication mappings, brand name associations, indication relationships, and anatomical structure connections. Side effect categorization encompasses common effects and post-marketing reported events with severity gradations and frequency classifications.

In an embodiment, medication relationship modeling connects RxNorm identifiers, Multum classifications, SNOMED CT concepts, and ICD-10 diagnostic codes through structured relationship frameworks. Drug therapeutic categories establish hierarchical classifications, while active ingredient mappings enable generic and brand name cross-referencing capabilities.

In an embodiment, an immunization ontology encompasses viral vaccine categories, drug component relationships, contraindication mappings, allergy associations, condition prerequisites, and severity reaction classifications. Patient scenarios incorporate age factors, gender considerations, National Drug Code identifiers, Current Procedural Terminology codes, and ICD-10 diagnosis codes for comprehensive vaccination decision support.

In an embodiment, a vaccine contraindication analysis processes patient conditions, including severe combined immunodeficiency disease, component allergies, and previous adverse reactions. Ontological reasoning enables dynamic contraindication identification based on patient-specific factors, vaccine composition data, and clinical guideline integration. Output generation provides contraindication explanations with supporting clinical rationale.

In an embodiment, a clinical data insights system provides semantic graph capabilities for healthcare provider queries about patient medication appropriateness, therapeutic alternatives, side effect profiles, contraindication identification, and care gap analysis. Query processing encompasses medication selection for specific conditions, adverse event identification, and treatment optimization recommendations.

In an embodiment, a medication appropriateness analysis determines patient suitability for beta blocker therapy, contraindications for vaccine administration, including MMR vaccines, travel vaccination requirements for specific geographic destinations, and alternative treatment options for patients with known allergies or contraindications.

In an embodiment, care gap identification processes encompass opioid-related treatment gaps, missing medication recommendations, duplicate medication detection, vaccination status verification, and condition-specific treatment requirements. Analysis capabilities extend to laboratory parameter monitoring, drug-laboratory interactions, and condition progression tracking.

In an embodiment, semantic reasoning capabilities support complex clinical queries, including medication ranking by likelihood of causing specific adverse effects, condition-medication interaction analysis, therapeutic alternative identification for contraindicated drugs, and comprehensive medication review for polypharmacy patients requiring multiple therapeutic interventions.

In an embodiment, an enterprise knowledge architecture addresses terminology concept integration, value set standardization, category establishment, and terminology system coordination across healthcare organizations. Variant identification processes encompass emerging medical conditions, experimental treatments, and evolving therapeutic approaches requiring systematic evaluation and integration.

In an embodiment, a knowledge asset management eliminates redundant terminologies, conflicting order sets, and inconsistent value set definitions through centralized governance processes. Labor reduction in knowledge creation relies on existing structural frameworks and client input mechanisms that streamline terminology development and maintenance workflows.

In an embodiment, an intelligent query system encompasses meaningful data connections, relationship discovery, and representation capabilities that support clinical decision-making processes. Computable artifact creation galvanizes decision support systems while improving data quality for patient care and research applications through standardized semantic frameworks.

### 6. PRACTICAL APPLICATIONS, ADVANTAGES, & IMPROVEMENTS

The embodiments provide substantial technical improvements in computer-based healthcare systems and EHR networks. By leveraging LLMs to process clinical guidance and generate structured pathways, the system significantly enhances the efficiency and accuracy of medical information processing within EHR networks. The automated pathway generation reduces the manual effort required to translate verbose clinical guidelines into actionable care plans, minimizing human error and inconsistencies across healthcare providers. The integration of these AI-generated pathways into EHR systems streamlines the flow of up-to-date, evidence-based practices directly into patient charts, improving the speed and quality of clinical decision-making at the point of care. The system's ability to codify pathways using standard medical terminologies enhances interoperability across different healthcare IT systems, facilitating seamless data exchange and reducing communication barriers between healthcare providers. Furthermore, the real-time analysis of patient charts to identify documentation gaps represents a significant advancement in maintaining comprehensive and accurate medical records, crucial for both patient care and administrative processes, such as billing and compliance. The technical improvements offered by these embodiments lead to more efficient healthcare delivery, reduced cognitive load on healthcare providers, and improved patient outcomes through consistent application of best practices across healthcare networks.

### 7. MISCELLANEOUS; EXTENSIONS

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

### 8. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

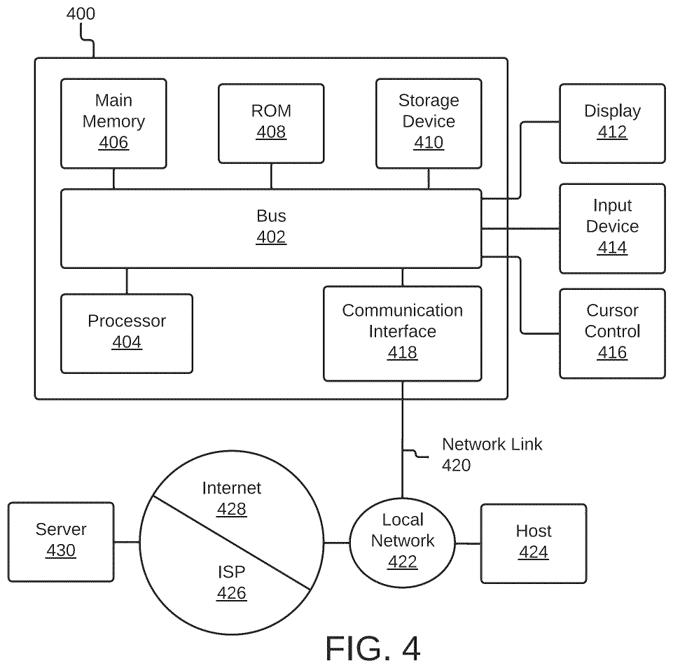

For example, FIG. 4 is a block diagram that illustrates a computer system 400 upon which an embodiment of the disclosure may be implemented. Computer system 400 includes a bus 402 or other communication mechanism for communicating information, and a hardware processor 404 coupled with bus 402 for processing information. Hardware processor 404 may be, for example, a general-purpose microprocessor.

Computer system 400 also includes a main memory 406, such as a random-access memory (RAM) or other dynamic storage device, coupled to bus 402 for storing information and instructions to be executed by processor 404. Main memory 406 also may be used for storing temporary variables or other intermediate information during execution of instructions to be executed by processor 404. Such instructions, when stored in non-transitory storage media accessible to processor 404, render computer system 400 into a special-purpose machine that is customized to perform the operations specified in the instructions.

Computer system 400 further includes a read-only memory (ROM) 408 or other static storage device coupled to bus 402 for storing static information and instructions for processor 404. A storage device 410, such as a magnetic disk or optical disk, is provided and coupled to bus 402 for storing information and instructions.

Computer system 400 may be coupled via bus 402 to a display 412, such as a cathode ray tube (CRT), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane.

Computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor 404 executing one or more sequences of one or more instructions contained in main memory 406. Such instructions may be read into main memory 406 from another storage medium, such as storage device 410. Execution of the sequences of instructions contained in main memory 406 causes processor 404 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 410. Volatile media includes dynamic memory, such as main memory 406. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid-state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may optionally be stored on storage device 410 either before or after execution by processor 404.

Computer system 400 also includes a communication interface 418 coupled to bus 402. Communication interface 418 provides a two-way data communication coupling to a network link 420 that is connected to a local network 422. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN. Wireless links may also be implemented. In any such implementation, communication interface 418 sends and receives electrical, electromagnetic or optical signals that carry digital data streams representing various types of information.

Network link 420 typically provides data communication through one or more networks to other data devices. For example, network link 420 may provide a connection through local network 422 to a host computer 424 or to data equipment operated by an Internet Service Provider (ISP) 426. ISP 426 in turn provides data communication services through the worldwide packet data communication network now commonly referred to as the “Internet” 428. Local network 422 and Internet 428 both use electrical, electromagnetic or optical signals that carry digital data streams. The signals through the various networks and the signals on network link 420 and through communication interface 418, which carry the digital data to and from computer system 400, are example forms of transmission media.

Computer system 400 can send messages and receive data, including program code, through the network(s), network link 420 and communication interface 418. In the Internet example, a server 430 might transmit a requested code for an application program through Internet 428, ISP 426, local network 422 and communication interface 418.

The received code may be executed by processor 404 as it is received, and/or stored in storage device 410, or other non-volatile storage for later execution.

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (NAT). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (HTTP). The requests are communicated through an interface, such as a client interface (such as a web browser), a program interface, or an application programming interface (API).

What is claimed is:

1. A non-transitory computer readable medium comprising instructions which, when executed by one or more hardware processors, causes performance of operations comprising:
receiving clinical guidance in digital text form;
generating a pathway from the clinical guidance using a large language model (LLM), wherein the generated pathway is a structured summary for a specific disease state organized into logical steps, including one or more of treatment goals, management strategies, and complication prevention measures; and
producing a patient chart in an electronic health record (EHR) system with information derived from the pathway.

2. The non-transitory computer readable medium of claim 1, wherein the operations further comprise:
providing the large language model with the clinical guidance and a context including one or more of pathway templates, pathway examples and pathway standards.

3. The non-transitory computer readable medium of claim 1, wherein the operations further comprise:
generating non-pathway data using the large language model and providing the EHR the non-pathway data.

4. The non-transitory computer readable medium of claim 1, wherein the operations further comprise:
codifying the pathway using standard medical terminologies for concepts with specific codes.

5. The non-transitory computer readable medium of claim 1, wherein the operations further comprise:
creating a graph representation from the pathway, wherein the graph representation is provided to the EHR system for the patient chart.

6. The non-transitory computer readable medium of claim 1, wherein the operations further comprise:
receiving patient information from the patient chart in the EHR system;
parsing the patient information using the large language model; and
generating a care recommendation based on a comparison of the parsed patient.

7. The non-transitory computer readable medium of claim 6, wherein generating the care recommendation comprises one or more of:
suggesting alternative medications considering one or more of condition, diagnosis, and known allergies,
optimizing treatment options based on factors such as cost or side effects, and
identifying potential gaps in documentation crucial for accurate diagnosis and proper reimbursement.

8. The non-transitory computer readable medium of claim 7, wherein the operations further comprise:
presenting the care recommendation through a user interface; and
distinguishing between information derived from established guidelines and insights generated by the large language model.

9. The non-transitory computer readable medium of claim 6, wherein the operations further comprise:
refining the care recommendation based on clinician interactions with the system implementing a feedback loop mechanism to continuously.

10. The non-transitory computer readable medium of claim 1, wherein the operations further comprise:
analyzing unstructured text in clinical notes of the patient chart using the large language model to extract relevant clinical concepts.

11. The non-transitory computer readable medium of claim 1, wherein the operations further comprise:
generating an immunization forecast by validating existing vaccine doses and suggesting missing vaccinations based on standard schedules or specific requirements.

12. The non-transitory computer readable medium of claim 1, wherein producing the patient chart comprises:
analyzing the patient chart in real-time to identify potential documentation gaps; and
prompting clinicians to address these gaps during a patient visit.

13. The non-transitory computer readable medium of claim 1, wherein the operations further comprise:
providing a suggested differential diagnosis.

14. A method comprising:
receiving clinical guidance in digital text form;
generating a pathway from the clinical guidance using a large language model (LLM), wherein the generated pathway is a structured summary for a specific disease state organized into logical steps, including one or more of treatment goals, management strategies, and complication prevention measures; and
producing a patient chart in an electronic health record (EHR) system with information derived from the pathway, wherein the method is performed by at least one device including a hardware processor.

15. The method of claim 14, wherein the operations further comprise:
providing the large language model with the clinical guidance and a context including one or more of pathway templates, pathway examples and pathway standards.

16. The method of claim 14, wherein the operations further comprise:
generating non-pathway data using the large language model and providing the EHR the non-pathway data.

17. The method of claim 14, wherein the operations further comprise:
codifying the pathway using standard medical terminologies for concepts with specific codes.

18. The method of claim 14, wherein the operations further comprise:
creating a graph representation from the pathway, wherein the graph representation is provided to the EHR system for the patient chart.

19. The method of claim 14, wherein the operations further comprise:
receiving patient information from the patient chart in the EHR system;
parsing the patient information using the large language model; and
generating a care recommendation based on a comparison of the parsed patient.

20. A system comprising:
at least one device including a hardware processor;
the system being configured to perform operations comprising:
one or more hardware processors, causes performance of operations comprising:
receiving clinical guidance in digital text form;
generating a pathway from the clinical guidance using a large language model (LLM), wherein the generated pathway is a structured summary for a specific disease state organized into logical steps, including one or more of treatment goals, management strategies, and complication prevention measures; and
producing a patient chart in an electronic health record (EHR) system with information derived from the pathway.
